(19) United States
(12) Patent Application Publication
(10) Pub. No.: US 2013/0012675 A1
(43) Pub. Date: Jan. 10, 2013

(54) LUBRICANT COMPONENT

(75) Inventor: Steven Dale Ittel, Wilmington, DE (US)

(73) Assignee: E.I. Dupont de Nemours and Company, Wilmington, DE (US)

(21) Appl. No.: 13/636,228

(22) PCT Filed: Mar. 28, 2011
(86) PCT No.: PCT/US11/30129

§ 371 Date: Sep. 20, 2012

Related U.S. Application Data
(60) Provisional application No. 61/390,365, filed on Oct. 6, 2010.
(60) Provisional application No. 61/357,362, filed on Jun. 22, 2010.
(60) Provisional application No. 61/318,570, filed on Mar. 29, 2010.

(51) Int. Cl. C08F 210/02; C07C 11/00

(52) U.S. Cl. 526/348; 585/17

(57) ABSTRACT

A lubricant component is an oligomer of a series α-olefins, made by forming a series of α-olefins by oligomerization of ethylene using an ethylene oligomerization catalyst, and then oligomerizing the series of α-olefins using a Lewis acid catalyst. The α-olefin oligomer, which often has a high Viscosity Index, may be used for example in a lubricant as the base oil or a viscosity index modifier. The α-olefin oligomer may also be a component of a lubricant additive, meant to be added to an already formulated lubricant to improve the lubricant's properties.

### CROSS-REFERENCE TO RELATED APPLICATIONS

This application claims the benefit of priority of U.S. Provisional Application Nos. 61/318,570 filed on Mar. 29, 2010; 61/357,362 filed on Jun. 22, 2010 and 61/390,365 filed on Oct. 6, 2010 which are herein incorporated by reference in their entirety.

### FIELD OF THE INVENTION

A lubricant or lubricant additive which contains a polyolefin which is made by contacting an ethylene oligomerization catalyst with ethylene to form a series of α-olefins, and then oligomerizing those α-olefins using a Lewis acid catalyst.

### TECHNICAL BACKGROUND

Lubricants are most commonly used to reduce friction between two moving parts in “contact” with each other, reducing wear of those parts, reducing corrosion of parts particularly metal parts, damping shock particularly in gears and forming seals as in between piston rings and engine cylinders. Probably the most common type of lubricant is used for machinery where metal, plastic, ceramic, etc. parts that rub against each other may be present in items such as internal combustion engines, transmissions, bearing assemblies, etc., but lubricants have other uses, for example in cosmetics.

Many lubricant compositions have a variety of ingredients in them, including heat stabilizers to prevent thermal degradation, antioxidants, viscosity index improvers, detergents, dispersants, pour-point depressants, friction modifiers, demulsifiers, corrosion inhibitors, etc. Many of these additives and other ingredients are described in Morteier et al., Chemistry and Technology of Lubricants,” 2^{nd} Ed., London, Springer (1996) and Leslie R. Rudnick, Lubricant additives”: Chemistry and Applications,” New York, Marcel Dekker (2003), both of which are hereby incorporated by reference. For lubricants that have to be useful over wide temperature ranges, such as internal combustion or jet engines, or that are exposed to a wide range of ambient temperatures, it is important that the viscosity of the lubricant change little with temperature. This is often referred to as the “Viscosity Index” (“VI”), and a higher number indicates less change in the viscosity as the temperature rises (this is usually desired).

Two of the major polymeric ingredients that may have a high VI are typically “base oils,” which are often the ingredient present in the largest amount, and “viscosity index improvers.” These oligomeric or polymeric materials are generally classified into groups, and one group of such polymeric materials is Group IV, “polyalphaolefins,” which typically have high Vls. These are polymers or oligomers of one or more α-olefins of the formula H₃C(CH₂)_{y}CH═CH₂ wherein y is about 5 to about 27 (this varies a bit). In some instance the alkyl groups in the α-olefin may be branched.

U.S. Pat. No. 3,780,128 describes making certain lubricant components by oligomerizing olefins using “Friedel-Crafts catalysts” (Lewis acids). Ethylene oligomerization processes with specific Schulz-Flory constants are not mentioned.

U.S. Pat. Nos. 2,183,503, 2816,944, 3,382,291, 3,652,706, 3,742,082, 3,763,244, 3,842,134, 2,620,365, 3,450,786, and 3,330,883 describe the use of Lewis acids to catalyze the oligomerization of olefins to olefin oligomers. Many of these patents also mention the use of these oligomers as lubricant components. None of these patents mentions the two step process described herein.

### SUMMARY OF THE INVENTION

This invention concerns a lubricant or a lubricant additive comprising an α-olefin oligomer made by a process comprising:

(1) contacting under ethylene oligomerizing conditions an ethylene oligomerization catalyst which oligomerizes ethylene to a series of α-olefins having the formula H(CH₂CH₂)ₙ CH═CH₂ wherein n is an integer of one or more and said ethylene oligomerization catalyst has a Schulz-Flory constant of about 0.45 to about 0.95; and then

(2) contacting said series of α-olefins with a Lewis acid to oligomerize said series of α-olefins to an α-olefin oligomer; and then

(3) optionally modifying said α-olefin oligomer to improve its properties for use in said lubricant or lubricant additive.

### DETAILS OF THE INVENTION

Herein certain terms are used and some of these are defined below:
By a “ethylene oligomerization catalyst comprising a transition metal” is meant a catalyst which comprises a transition metal of Groups 3-12 (IUPAC notation) and the lanthanides, such Zr, Hf, V, Ti, etc. These types of catalysts are well known in the field of making α-olefins, see for instance J-C. Wasilke et al., Chem. Rev., vol. 105, p. 1001-1020 (2005), which is hereby incorporated by reference, especially pages 1013-1015, and references cited below for certain complexes of the ligand (I). In one preferred form the ethylene oligomerization catalyst comprises a transition metal.

By an “α-olefin” is meant a compound of the formula H(CH₂CH₂)ₙCH═CH₂ wherein n is an integer of 1 or more.

By a “series” of α-olefins is meant compounds having the formula H(CH₂CH₂)ₙCH═CH₂ wherein at least three, more preferably 4, and very preferably 5, compounds having different n values are produced, and n is an integer of 1 or more. Preferably at least three of these values are 1, 2, and 3. Preferably in this series of α-olefins in some of the α-olefins n is 3 or more.

By “hydrocarbyl group” is meant a univalent group containing only carbon and hydrogen. As examples of hydrocarbyls may be mentioned unsubstituted alkyls, cycloalkyls and aryls. If not otherwise stated, it is preferred that hydrocarbyl groups (and alkyl groups) herein contain from 1 to about 30 carbon atoms.

By “substituted hydrocarbyl” herein is meant a hydrocarbyl group that contains one or more substituent groups that are inert under the process conditions to which the compound containing these groups is subjected (e.g., an inert functional group, see below). The substituent groups also do not substantially detrimentally interfere with the polymerization process or the operation of the polymerization catalyst system. If not otherwise stated, it is preferred that (substituted) hydrocarbyl groups herein contain from 1 to about 30 carbon atoms. Included in the meaning of “substituted” are rings containing one or more heteroatoms such as nitrogen, oxygen and/or sulfur, and the free valence of the substituted hydrocarbyl may be to the heteroatom. In a substituted hydrocarbyl, all of the hydrogens may be substituted, as in trifluoromethyl.

By an “(inert) functional group” herein is meant a group, other than hydrocarbyl or substituted hydrocarbyl, that is inert under the process conditions to which the compound containing the group is subjected. The functional groups also do not substantially deleteriously interfere with any process described herein that the compound in which they are present may take part in. Examples of functional groups include halo (fluoro, chloro, bromo and iodo), and ether such as —OR⁵⁰ wherein R⁵⁰ is hydrocarbyl or substituted hydrocarbyl. In cases in which the functional group may be near a transition metal atom, the functional group alone should not coordinate to the metal atom more strongly than the groups in those compounds that are shown as coordinating to the metal atom, that is, they should not displace the desired coordinating group.

By a “cocatalyst” or a “catalyst activator” is meant one or more compounds that react with a transition metal compound to form an activated catalyst species. One such catalyst activator is an “alkylaluminum compound,” which herein means a compound in which at least one alkyl group is bound to an aluminum atom. Other groups such as, for example, alkoxide, hydride, an oxygen atom bridging two aluminum atoms, and halogen may also be bound to aluminum atoms in the compound.

The “Schulz-Flory constant” (“SFC”) of the mixtures of α-olefins produced is a measure of the molecular weights of the olefins obtained, usually denoted as factor K, from the Schulz-Flory theory (see B. Elvers, et al., Ed. Ullmann's Encyclopedia of Industrial Chemistry, Vol. A13, VCH Verlagsgesellschaft mbH, Weinheim, 1989, p. 243-247 and 275-276). This is defined as:

K=n(Cₙ₊₂olefin)/n(Cₙolefin)

wherein n(Cₙ olefin) is the number of moles of olefin containing n carbon atoms, and n(Cₙ₊₂ olefin) is the number of moles of olefin containing n+2 carbon atoms, or in other words the next higher oligomer of Cₙ olefin. From this can be determined the weight (mass) and/or mole fractions of the various olefins in the resulting oligomeric reaction product mixture.

By “aryl” is meant a monovalent aromatic group in which the free valence is to the carbon atom of an aromatic ring. An aryl may have one or more aromatic rings, which may be fused, connected by single bonds or other groups.

By “substituted aryl” is meant a monovalent aromatic group substituted that contains one or more substituent groups that are inert under the process conditions to which the compound containing these groups is subjected (e.g., an inert functional group, see below). The substituent groups also do not substantially detrimentally interfere with the polymerization process or operation of the polymerization catalyst system. If not otherwise stated, it is preferred that (substituted) aryl groups herein contain from 1 to about 30 carbon atoms. Included in the meaning of “substituted” are rings containing one or more heteroatoms, such as nitrogen, oxygen and/or sulfur, and the free valence of the substituted hydrocarbyl may be to the heteroatom. In a substituted aryl all of the hydrogens may be substituted, as in trifluoromethyl. These substituents include (inert) functional groups. Similar to an aryl, a substituted aryl may have one or more aromatic rings, which rings may be fused or connected by single bonds or other groups; however, when the substituted aryl has a heteroaromatic ring, the free valence in the substituted aryl group can be to a heteroatom (such as nitrogen) of the heteroaromatic ring instead of a carbon.

By “process conditions” herein is meant conditions for producing the series of α-olefins, whether in the presence of the copolymerization catalyst or not. Such conditions may include temperature, pressure, and/or oligomerization method such as liquid phase, continuous, batch, and the like. Also included may be cocatalysts that are needed and/or desirable. If in the presence of the copolymerization catalyst, the SFC is measured under conditions in which the copolymerization catalyst is not present.

By a “Lewis acid” is meant the classic definition of a Lewis acid, a compound that may accept a pair of electrons from a Lewis base to form an adduct. In some α-olefin oligomerization patent literature these compounds are sometimes referred as “Friedel-Crafts catalysts,” but are more correctly called Lewis acids. Useful Lewis acids include AlCl₃, boron trifluoride, FeCl₃, etc. Boron trifluoride and AlCl₃ are preferred Lewis acids, and boron trifluoride is more preferred. In another preferred form the Lewis acid is aprotic, that is not acidic because of a hydronium ion.

It is to be understood that “ethylene oliogomerization catalyst” may also include other compounds such as cocatalysts and/or other compounds normally used with the oliogomerization catalyst and/or copolymerization catalyst to render that particular catalyst active for the polymerization or oligomerization it is meant to carry out. Preferably the ethylene oligomerization catalyst comprises a complex of a transition metal.

A preferred oligomerization catalyst is an iron complex of a ligand of the formula:

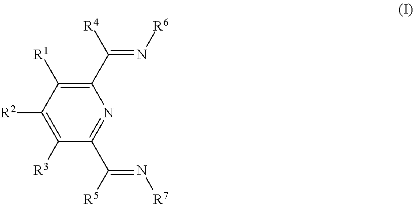

wherein: R¹, R² and R³ are each independently hydrogen, hydrocarbyl, substituted hydrocarbyl or an inert functional group, provided that any two of R¹, R², and R³ vicinal to one another, taken together may form a ring; R⁴ and R⁵ are each independently hydrogen, hydrocarbyl, substituted hydrocarbyl or an inert functional group, provided that R¹ and R⁴ and/or R³ and R⁵ taken together may form a ring; and R⁶ and R⁷ are each independently aryl or substituted aryl.

In an iron complex of (I), (I) is usually thought of as a tridentate ligand coordinated to the iron atom through the two imino nitrogen atoms and the nitrogen atom of pyridine ring. It is generally thought that the more sterically crowded it is about the iron atom the higher the molecular weight of the polymerized olefin (ethylene). In order to make α-olefins, and especially to make in a process the desired SFC (such as 0.40-0.95) very little steric crowding about the iron atom is desired.

Such compounds of (I) are readily available. In WO2005/092821 it is demonstrated that the iron complex in which R⁴ and R⁵ are both hydrogen, and R⁶ and R⁷ are both phenyl, has a SFC of about 0.29 (this reference states the SFC is about 0.4, but this apparently based incorrectly on the weight fraction of the olefins produced, not correctly mole fraction]. In G. J. P. Britovsek et al., Chem. Eur. J., vol. 6 (No. 12), p. 2221-2231 (2000), which is hereby incorporated by reference, a ligand in which R⁴ and R⁵ are both hydrogen and R⁶ and R⁷ are both 2-methylphenyl, gives an oligomerization at 50° C. in which the SFC is reported to be 0.50. Other combinations of groups would give ligands with useful relatively low SFCs. For instance, R⁴ and R⁵ may both be methyl or hydrogen (or one could be methyl and one could be hydrogen) and R⁶ could be phenyl, while R⁷ could be 2-fluorophenyl or 2-methylphenyl or 2-chlorophenyl; or R⁶ and R⁷ could both be 2-fluorophenyl; or R⁶ and R⁷ could both be 4-isopropylphenyl; or both R⁶ and R⁷ could both be 4-methylphenyl. Other variations in which just small increments of steric hindrance are added or subtracted about the iron atom are obvious to those skilled in the art. It is also believed that in addition to these steric effects that electron-withdrawing groups on R⁶ and/or R⁷ tend to lower the SFC.

For “moderate” SFCs, those in the approximate range of about 0.55 to about 0.70, R⁴ and R⁵ may both be methyl and R⁶ and R⁷ may both be 2-methylphenyl or 2-ethylphenyl, or R⁴ and R⁵ may both be methyl and R⁶ may be 2,6-dimethylphenyl and R⁷ may phenyl. See for U.S. Pat. Nos. 6,103,946, 7,049,442 and 7,053,020, all of which are hereby incorporated by reference.

For higher SFCs somewhat more sterically crowded complexes can be used. R⁴ and R⁵ may both be methyl and R⁶ may both be 2,6-dimethylphenyl and R⁷ may be 2-methylphenyl, or R⁴ and R⁵ may both be methyl and R⁶ may be 2,6-diisopropyllphenyl and R⁷ may 2-isopropylphenyl.

The synthesis of the ligands (I) and their iron complexes are well known, see for instance U.S. Pat. Nos. 6,103,946, 7,049,442 and 7,053,020, G. J. P. Britovsek, et al., cited above, and World Patent Application WO2005/092821, World Patent Applications 1999/012981 and 2000/050470, all of which are hereby incorporated by reference.

Other relatively small aryl groups may also be used, such as 1-pyrrolyl, made from substituted or unsubstituted 1-aminopyrrole (see World Patent Application 2006/0178490, which is hereby incorporated by reference). Analogous substitution patterns to those carried out in phenyl rings may also be used to attain the desired degree of steric hindrance, and hence the desired SFC. Aryl groups containing 5-membered rings such as 1-pyrrolyl may especially useful for obtaining low SFCs, since they are generally less sterically crowding than 6-membered rings. Preferred aryl groups for R⁶ and R⁷ are phenyl and substituted phenyl.

While steric hindrance about the iron atom is usually the dominant item controlling the Schulz-Flory constant, process conditions may have a lesser effect. Higher process temperatures generally give lower SFCs, while higher ethylene pressures (concentrations) generally give a higher SFC, all other conditions being equal. In order to measure the SFC of the oligomerization during the manufacture of the branched polyethylene the process is carried out using the same conditions as the process to produce the branched polyethylene, but the copolymerization catalyst is omitted and any cocatalysts are scaled back in relationship to the total amount of oliogomerization catalyst present compared to the total of the copolymerization catalyst and oligomerization catalyst usually used. However it is to be noted that somewhat more than normal cocatalyst, such as an alkylaluminum compound, may have to be used to remove traces of any process poisons present such as water.

To determine the SFC, the resulting mixture of α-olefins is analyzed to determine their molecular ratios. This is most conveniently done by standard gas chromatography using appropriate standards for calibration. Preferably the ratios (as defined by the equation for “K,” above) between olefins from C₄ to C₁₂ are each measured and then averaged to obtain the SFC. If the ratios of higher olefins, such as C₁₂/C₁₀ are too small to measure accurately, they may be omitted from the calculation of the constant.

The choice of the desired SFC is somewhat complex. It is believed that to achieve a relatively high VI the branches on the polymer should be relatively long, but if the branches are very long they themselves may have a tendency to crystallize, thereby possibly having a deleterious effect on low temperature properties such as pour point. Very long branches may also increase the molecular weight of the α-olefin oligomer to the point where its viscosity is too high. Short branches are believed to be relatively ineffective in increasing VI. Therefore the desired SFC will often be a compromise between these and other factors. The higher the SFC the larger the proportion of relatively long chain α-olefins produced, and hence long branches incorporated into the polyolefin. The lower the SFC the relatively higher amount of short chain α-olefins produced and the short branches incorporated into the polyolefin. A preferred SFC range is about 0.50 to about 0.90, more preferably about 0.55 to about 0.85.

It is preferred that the α-olefins produced in the ethylene oligomerization process be a mixture of relatively pure compounds of the formula H(CH₂CH₂)ₙCH═CH₂ wherein n is an integer of 1 or more. This can be judged by analyzing the fraction of the series of α-olefins which have 12 carbon atoms. The “desired” compound is 1-dodecene, but this fraction may also contain, for instance, dodecane or other saturated alkanes containing 12 carbon atoms, linear dodecenes wherein the olefinic bond in internal, and branched dodecenes in which the olefinic bond it terminal or internal. The purity of this fraction is determined by careful gas chromatography of this fraction. Using standards the elution time of the 1-dodecene, and other compounds if desired, is determined, and then the molar amount of 1-dodecene present in this fraction is taken as the area percent (or signal strength) of the peak 1-dodecene of the total C₁₂ fraction. That is

mole % 1-dodecene=[(area 1-dodecene)/(total area)]ₓ₁₀₀.

Preferably the mole % 1-dodecene in this faction is at least 80%, more preferably at least 85%, very preferably 90% and especially preferably 93%.

After the ethylene oligomerization is done the stream of the series of α-olefins can be treated in a number of ways for instance solvent may be removed, the oligomerization catalyst be deactivated, or the stream of α-olefins be partially fractioned to remove, for instance, lower boiling compounds, such as 1-butene and perhaps 1-hexene. It is preferred that if lower boiling compounds are removed at least half of the 1-octene is present after fractionation, compared to the amount of 1-dodcene present (determined by gas chromatography using appropriate standards) before and after fractionation. The α-olefin stream is preferably added as a liquid to the α-olefin oligomerization part of the process.

The oligomerization of the series of α-olefins can be carried out by methods well known in the art, see for instance U.S. Pat. Nos. 2,183,503, 2816,944, 3,382,291, 3,652,706, 3,742,082, 3,763,244, 3,842,134, 2,620,365, 3,450,786, and 3,330,883, which are hereby incorporated by reference. The particular Lewis acid used, the process conditions such as temperature, olefin concentration, time of reaction, ratio of Lewis acid to α-olefin and other conditions will determine the exact nature of the oligomerized α-olefin produced. These effects are illustrated in the above-listed patents and many other documents on the Lewis acid catalyzed oligomerization of α-olefins, many of them directed to make such oligomers for use in lubricants. The α-olefin oligomers made herein are cooligomers, oftentimes most of the molecules in these oligomers being made from two or more α-olefins having a differing number of carbon atoms. The structures of the individual α-olefin cooligomers tend to be complex, not only because of the variety of structures which are inherently produced in such a reaction, but also because 2 or more different α-olefins may be combined to form a cooligomer molecule.

The Mn (number average molecular weight) of the oligomerized α-olefins is preferably in the range of about 300 to about 5,000. The Mn is measured by standard methods using Size Exclusion Chromatography (sometimes called Gel Permeation Chromatography) using a linear polyethylene standard. A more preferred minimum Mn is about 500, especially preferably about 1000. A more preferred maximum Mn is about 3,000, more preferably about 2,000 and very preferably about 1,000. It is to be understood that any preferred minimum Mn may be combined with any preferred maximum Mn to form a preferred Mn range for the polyolefin. The molecular weight of the polyolefin may be controlled by the oligomerization conditions.

It is preferred that the α-olefin oligomer made herein, directly from the α-olefin oligomerization and/or after modification, have a Viscosity Index of about 125 or more, more preferably about 140 or more, and very preferably about 150 or more. Viscosity Index is measured by ASTM Method D2270-04.

After the α-olefin oligomer has been formed it may undergo treatment, chemical and/or physical to make more suitable component in a lubricant. In most cases it would be desirable to remove any solvent or other liquid from the α-olefin oligomer formed in the α-olefin oligomerization process, and to remove, to the practical extent possible any unreacted α-olefins in the product. Both of these may be accomplished by distilling or otherwise volatilizing the solvent and α-olefins. Other treatments may also be done, for instance it may be fractionated so that only a certain molecular weight portion is used, and/or it may be hydrogenated to remove unsaturation, and/or treated with activated carbon to remove color, and/or polar compounds be grafted to the α-olefin oligomer (usually at the site of residual double bonds). The latter is particularly useful for forming dispersants. Other similar treatments known for α-olefin oligomers known in the art may also be used. If suitable the α-olefin oligomer may be used without post treatment in a lubricant or lubricant additive.

Formerly α-olefin oligomers for use in lubricants were made from previously synthesized, and often purified, α-olefins, such as 1-octene, and/or 1-decene and/or 1-dodecene. These olefins are significantly more expensive than ethylene from which they are usually made. The present process makes the olefins which are oligomerized without (much) purification. This saves considerable cost in the manufacture of the α-olefin oligomer.

In lubricants, the α-olefin oligomers of the present invention are particularly useful as a base oil or a viscosity index improver, or for other uses as noted above. The present polyolefin may be part of a lubricant additive that improves the VI of an already formulated lubricant. Use as a base for the lubricant may also help improve the lubricant VI.

1-11. (canceled)

12. A lubricant or a lubricant additive, comprising an α-olefin oligomer made by a process comprising:
(1) contacting, under ethylene oligomerizing conditions, an ethylene oligomerization catalyst that oligomerizes ethylene to a series of α-olefins having the formula H(CH₂CH₂)ₙCH═CH₂, wherein n is an integer of one or more, and said ethylene oligomerization catalyst has a Schulz-Flory constant of about 0.45 to about 0.95; and then
(2) contacting said series of α-olefins with a Lewis acid to oligomerize said series of α-olefins to an α-olefin oligomer; and then
(3) optionally modifying said α-olefin oligomer to improve its properties for use in said lubricant or lubricant additive.

13. The lubricant or lubricant additive as recited in claim 12 wherein said ethylene oligomerization catalyst has a Schulz-Flory constant of about 0.55 to about 0.85.

14. The lubricant or lubricant additive as recited in claim 12 wherein said oligomerization catalyst is an iron complex of a ligand of the formula: 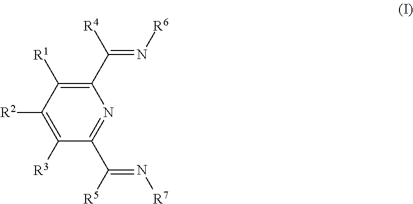
wherein: R¹, R², and R³ are each independently hydrogen, hydrocarbyl, substituted hydrocarbyl, or an inert functional group, provided that any two of R¹, R², and R³ vicinal to one another, taken together may form a ring; R⁴ and R⁵ are each independently hydrogen, hydrocarbyl, substituted hydrocarbyl, or an inert functional group, provided that R¹ and R⁴ and/or R³ and R⁵ taken together may form a ring; and R⁶ and R⁷ are each independently aryl or substituted aryl.

15. The lubricant or lubricant additive as recited in claim 14 wherein said oligomerization catalyst has a Schulz-Flory constant of about 0.55 to about 0.85.

16. The lubricant or lubricant additive as recited in claim 12 wherein said Lewis acid a an aprotic Lewis acid.

17. The lubricant or lubricant additive as recited in claim 12 wherein said Lewis acid is aluminum chloride or boron trifluoride.

18. The lubricant or lubricant additive as recited in claim 14 wherein said Lewis acid a an aprotic Lewis acid.

19. The lubricant or lubricant additive as recited in claim 15 wherein said Lewis acid is aluminum chloride or boron trifluoride.

20. The lubricant or lubricant additive as recited in claim 12 wherein said α-olefin oligomer has a number average molecular weight of about 300 to about 5,000.

21. The lubricant or lubricant additive as recited in claim 14 wherein said polyolefin has a number average molecular weight of about 300 to about 5,000.

22. The lubricant or lubricant additive as recited in claim 12 wherein said α-olefin oligomer is hydrogenated.

23. The lubricant or lubricant additive as recited in claim 14 wherein said α-olefin oligomer is hydrogenated.

24. The lubricant or lubricant additive as recited in claim 12 wherein said α-olefin oligomer is a base oil.

25. The lubricant or lubricant additive as recited in claim 14 wherein said α-olefin oligomer is a base oil.

26. The lubricant or lubricant additive as recited in claim 12 wherein said α-olefin oligomer is a viscosity index modifier.

27. The lubricant or lubricant additive as recited in claim 14 wherein said α-olefin oligomer is a viscosity index modifier.

28. The lubricant or lubricant additive as recited in claim 12 wherein said α-olefin oligomer has a viscosity index of about 125 or more.

29. The lubricant or lubricant additive as recited in claim 14 wherein said α-olefin oligomer has a viscosity index of about 125 or more.
